(19) United States
(12) Patent Application Publication
(10) Pub. No.: US 2023/0271372 A1
(43) Pub. Date: Aug. 31, 2023

(54) DEVICE FOR TREATING CONTAINER BLANKS

(71) Applicant: KRONES AG, Neutraubling (DE)

(72) Inventor: Alexander KAISER, Neutraubling (DE)

(21) Appl. No.: 18/007,427

(22) PCT Filed: Jul. 22, 2021
(86) PCT No.: PCT/EP2021/070540

§ 371 Date: Jan. 30, 2023

(30) Foreign Application Priority Data
Jul. 31, 2020 (DE) 10 2020 120 284.9

(51) Int. Cl. B29C 49/64; B29B 13/02; B29C 49/42; F27B 9/24; B65G 54/02; A61L 2/26; A61L 2/16; A61L 2/08

(52) U.S. Cl. CPC B29C 49/6418; B29B 13/02; B29C 49/42065; F27B 9/243; B65G 54/02; A61L 2/26; A61L 2/16; A61L 2/08; B29L 2031/7158

(57) ABSTRACT

The invention relates to a device for treating container blanks for a container treatment installation. The device comprises a furnace for heating the container blanks. A planar drive system has a base element and a plurality of movement devices for transporting the container blanks. The plurality of movement devices are movable independently from one another relative to the base element. The container blanks can be moved through the furnace by means of the plurality of movement devices.

### TECHNICAL FIELD

The invention relates to a device for treating container blanks for a container treatment machine.

### TECHNICAL BACKGROUND

Thin-walled plastics containers of thermoplastic material such as, for example, PET are usually produced from injection-molded preforms/container blanks and shaped into containers in a two-stage stretch blow molding method. During processing, the container blanks are heated to a defined process temperature in order to make possible the shaping operation during the stretch blow molding. The container blanks are thereby first heated in a heating section in order to heat the relatively thick-walled container blanks sufficiently over their entire cross section, so that a hollow body can be formed by the subsequent shaping process by means of a stretch molding method.

Devices for the blow molding of articles from prefabricated container blanks are known from the prior art, for example from DE 10 2010 018 153 A1.

Conventionally, the container blanks can be transported through the furnace by being pulled through on a chain. However, if container blanks have to be discarded upstream of the furnace because they are damaged, this can have the result that, in the case of transport by means of the chain, one or more large gaps between the container blanks are transported through the furnace. This can lead to the container blanks adjacent to the gap receiving more heat, and the blow molding process with such container blanks would become unstable. The container blanks next to the gap may thus have to be discarded after heating and before the blow molding process. This can have the result that the gap in the container blank stream/container stream becomes larger and has to be closed again in a technically complex manner.

The object underlying the invention is to provide an alternative and/or improved device for treating container blanks.

### SUMMARY OF THE INVENTION

The object is achieved by the features of independent claim 1. Advantageous further developments are indicated in the dependent claims and the description.

One aspect of the present disclosure relates to a device for treating container blanks for a container treatment installation (e.g. for producing, cleaning, testing, filling, capping, labeling, printing and/or packaging containers for liquid media, preferably drinks or liquid foods). The device has a furnace for heating the container blanks. The device has a planar drive system having a base element and a plurality of movement devices for transporting the container blanks. The plurality of movement devices are movable independently of one another relative to the base element, preferably by means of magnetic interaction between the base element and the plurality of movement devices. The container blanks can be moved through the furnace by means of the plurality of movement devices.

Advantageously, the device permits a more compact construction of the furnace. The planar drive system can be operated virtually without wear. The planar drive system additionally permits a flexible movement of the container blanks through the furnace. Gaps in the container blank stream can, for example, be closed in a flexible manner. A reject rate can thus preferably be reduced. The length of the heating section through the furnace can also be changed owing to the flexibility of the movement of the movement devices and thus, for example, can be adapted to a throughput or an output of the device.

Preferably, the movement devices can be moved outside the furnace, while the movement devices move the container blanks through the furnace.

In one exemplary embodiment, the base element is arranged above the furnace, and the plurality of movement devices can be moved upside down on an underside of the base element. Alternatively, the base element can be arranged beneath the furnace, for example, and the movement devices can be movable upright on an upper side of the base element.

In a further exemplary embodiment, the furnace has at least one heating channel which is open toward a longitudinal outer side (e.g. upper side, underside, left longitudinal outer side or right longitudinal outer side). The base element is arranged on the longitudinal outer side, preferably for moving the container blanks through the heating channel by means of the plurality of movement devices.

In a further exemplary embodiment, the plurality of movement devices each have at least one holder, preferably a (e.g. passive or active) mandrel. The at least one holder is configured to hold at least one container blank, preferably at a neck region of the container blank (e.g. internally or externally at the neck region).

In one embodiment, the at least one holder is designed to be changeable. Different holders for container blanks of different sizes and/or different shapes can thus advantageously be combined with the movement devices.

In a further embodiment, the device has a holder magazine which holds a plurality of different holders, wherein the plurality of movement devices can be moved to the holder magazine in order to change, preferably automatically, the at least one holder. Advantageously, a change of format of the container blanks can thus be facilitated considerably.

In a further embodiment, the at least one holder is rotatable by means of a rotation mechanism, preferably a mechanical rotation mechanism, of the respective movement device in order to rotate the at least one held container blank, preferably while it is being moved through the furnace. The rotation can preferably permit uniform or non-uniform heating of the container blanks. It is possible that the rotation is uniform or predeterminedly non-uniform. Predeterminedly non-uniform rotation can be used to purposively achieve warmer and colder regions of the container blank (so-called preferential heating). In particular bottles with a non-uniform cross section, that is to say, for example, square or oval bottles, can thus be produced in a simpler and better manner.

In one embodiment variant, the furnace has a rotation apparatus which is configured to rotate the rotation mechanism, preferably by means of interlocking engagement and/or force-based engagement, while the respective movement device is being moved along the rotation apparatus. Preferably, a separate drive for the rotation mechanism can thus be dispensed with. Instead, the rotation mechanism can be driven by the movement of the movement devices themselves.

Preferably, the rotation apparatus can extend at least in some portions along a heating channel of the furnace.

In a further embodiment variant, the rotation mechanism has a toothed portion, preferably a toothed wheel, and the rotation apparatus has a toothed portion, preferably a toothed rack, which mesh with one another in order to rotate the rotation mechanism.

In one exemplary embodiment, the rotation mechanism has a roller and the rotation apparatus has a rolling surface for the roller.

In a further exemplary embodiment, the rotation mechanism interacts with the rotation apparatus by means of magnetic force in order to rotate the rotation mechanism.

In one embodiment, the planar drive system is configured (e.g. by means of a control unit) to rotate the plurality of movement devices relative to the base element about their own vertical axis while the container blanks are being moved through the furnace, preferably in order to heat the container blanks uniformly or non-uniformly. This can make it possible, for example, to dispense with a separate rotation mechanism for the holder. It is possible that the rotation is uniform or predeterminedly non-uniform. A predeterminedly non-uniform rotation can be used purposively to achieve warmer and colder regions of the container blank (so-called preferential heating).

In a further embodiment, the planar drive system is configured (e.g. by means of a control unit) to carry out a stroke movement of the movement device relative to the base element when a container blank is transferred to a movement device or when a container blank is transferred from a movement device, so that preferably a holder of the movement device enters the container blank or emerges from the container blank.

In a further embodiment, the planar drive system is configured (e.g. by means of a control unit) to move a stream of container blanks transferred at non-uniform distances to the plurality of movement devices through the furnace at a uniform distance. It is thus possible, for example, to prevent a reject rate owing to excessive heating of some container blanks, which can be caused by excessively large gaps in the container blank stream.

In one embodiment variant, the planar drive system is configured (e.g. by means of a control unit) to adapt a length of a heating section of the movement of the container blanks through the furnace by means of the plurality of movement devices, preferably in dependence on a desired container blank throughput.

In a further embodiment variant, the planar drive system is configured (e.g. by means of a control unit) to adapt a residence time of the container blanks in the furnace by adapting a speed of the plurality of movement devices, preferably in dependence on a desired container blank throughput.

In one exemplary embodiment, the furnace has a plurality of preferably individually activatable heating channels through which the container blanks are movable in a flexible manner by means of the plurality of movement devices, preferably in dependence on a respective desired length of a heating section of the movement of the container blanks through the furnace.

In a further exemplary embodiment, the device further has a sterilization apparatus for sterilizing the container blanks, preferably by means of hydrogen peroxide or electromagnetic irradiation. The container blanks can preferably be moved through the sterilization apparatus by means of the plurality of movement devices, preferably downstream of the furnace. Advantageously, particularly hygienic container production can thus be made possible. It is also possible that the sterilization apparatus is a portion of the furnace, preferably at an outlet portion of the furnace.

In a further exemplary embodiment, the device further has a blow molding machine, preferably a stretch blow molding machine, for blow molding containers from the container blanks, wherein the blow molding machine is arranged downstream of the furnace and/or is configured for the transfer of container blanks from the plurality of movement devices. The transfer can be carried out directly, for example, or a transfer starwheel (e.g. a reduction starwheel), for example, can be arranged between the base element and the blow molding machine for the transfer of the container blanks.

Preferably, the term “control unit” can refer to an electronic system (e.g. with microprocessor(s) and memory) which, according to its configuration, can perform control functions and/or closed-loop control functions and/or processing functions. Also, when the term “control” is used herein, it can expediently include or mean also “closed-loop control” or “control with feedback” and/or “process”.

The above-described preferred embodiments and features of the invention can be combined with one another as desired.

### BRIEF DESCRIPTION OF THE FIGURES

Further details and advantages of the invention are described below with reference to the accompanying drawings, in which:

FIG. 1 is a schematic block diagram of a device for treating container blanks;

FIG. 2 is a schematic block diagram of a further device for treating container blanks;

FIG. 3 is a schematic block diagram of another further device for treating container blanks;

FIG. 4 is a schematic block diagram of another further device for treating container blanks;

FIG. 5 is a schematic plan view of another further device for treating container blanks;

FIG. 6 is a schematic side view of a portion of a planar drive system; and

FIG. 7 is a schematic view from beneath of a portion of a planar drive system.

The embodiments shown in the figures correspond at least in part, so that similar or identical parts are provided with the same reference numerals and reference is also made for the explanation thereof to the description of the other embodiments or figures, in order to avoid repetition.

### DETAILED DESCRIPTION OF EXEMPLARY EMBODIMENTS

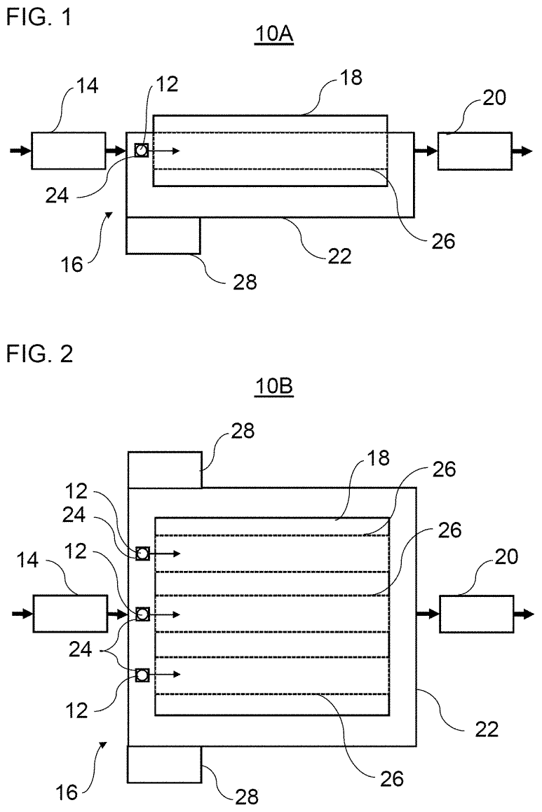

FIG. 1 shows a device 10A for treating container blanks 12. For reasons of clarity, only one container blank 12 is shown in FIG. 1. The device 10A has a feed conveyor 14, a planar drive system 16, a furnace 18, and a blow molding machine 20.

The feed conveyor 14 conveys container blanks 12 to the planar drive system 16. For example, a transfer starwheel, for example a sawtooth starwheel, of the feed conveyor 14 can transfer the container blanks 12 to the planar drive system 16. Further apparatuses can be arranged upstream (in respect of the container blank stream) of the feed conveyor 14, for example an inspection apparatus, which is able to discharge faulty container blanks 12 from the container blank stream, and a sorting apparatus, which can provide the container blanks 12 separately in a respective desired orientation in a container blank stream. If an inspection apparatus is present, it can happen during operation that the container stream supplied to the planar drive system 16 has non-uniform distances or gaps between the container blanks 12 transferred in succession. The non-uniform distances can be caused by the discharge of faulty container blanks 12.

The planar drive system 16 is arranged downstream of the feed conveyor 14. The planar drive system 16 is preferably a magnetic planar drive system.

The planar drive system 16 has a base element 22, a plurality of movement devices 24 (movers or rotors), and a control unit. For reasons of clarity, only one movement device 24 and no control unit is shown in FIG. 1.

The movement devices 24 can be moved over the base element 22 freely and independently of one another by means of magnetic interaction with the base element 22. The control unit is configured to control a movement of the movement devices 24 relative the base element 22.

The movement devices 24 can also be rotated (e.g. yawing) relative to the base element 22 by means of magnetic interaction with the base element 22. The movement devices 24 can also be inclined (e.g. pitching and/or rolling) relative to the base element 22 by means of magnetic interaction with the base element 22. The movement devices 24 can likewise perform a stroke movement upward or downward relative to the base element 22 by means of magnetic interaction with the base element 22. The number of movement devices 24 can be chosen freely according to the application.

The base element 22 forms a stator of the planar drive system 16. The movement devices 24 form rotors of the planar drive system 16. The movement devices 24 are carried in a contactless manner by the base element 22, preferably on an upper side or an underside of the base element 22. The base element 22 can have, for example, a plurality of electromagnets, for example electric coils, arranged in a distributed manner. The movement devices 24 can have permanent magnets. The electromagnets are preferably arranged in a matrix which extends in a plane of the base element 22 (here preferably the horizontal plane). The control unit of the planar drive system 16 can control a current supply to the electromagnets of the base element 22, in order to build up electromagnetic fields at a desired position of the base element 22 with a desired field strength. An advance, a rotation, an inclination and/or a stroke of the movement devices 24 can be effected by corresponding electromagnetic fields of the electromagnets of the base element 22. Preferably, the base element 22 is oriented in a horizontal plane. However, other orientations are also conceivable, for example an orientation inclined relative to the horizontal plane, preferably a vertical orientation.

The base element 22 can have different characteristics and shapes, for example strip-shaped, rectangular, square, polygonal, round, circular, etc. For cleanroom applications, the base element 22 can be arranged at least in part inside a cleanroom or can form a boundary wall, for example a lower boundary wall, of the cleanroom.

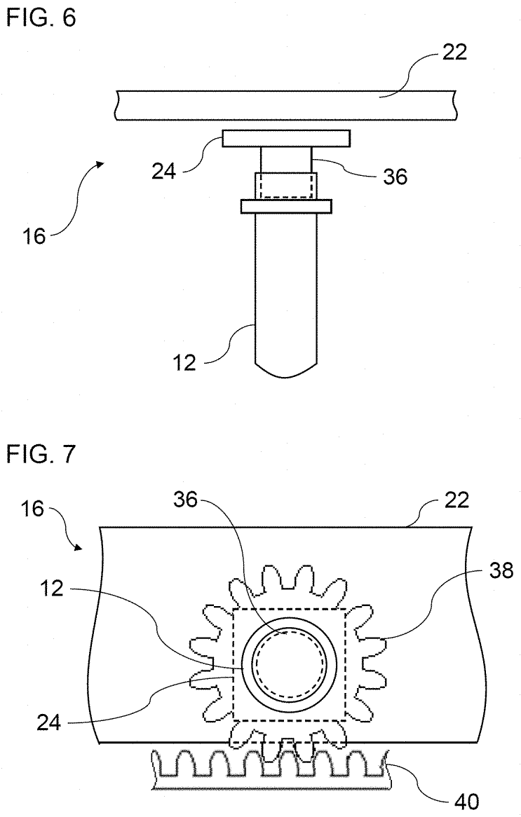

The movement devices 24 are configured to move the container blanks 12 when the respective movement device 24 moves relative to the base element 16. To that end, the movement devices 24 can each have, for example, at least one holder with which in each case at least one container blank 12 can be held. An exemplary embodiment of a holder is described herein with reference to FIG. 6.

The container blanks 12 can be transported by the feed conveyor 14 (for example belt conveyor or transfer starwheel) to the base element 22 or the movement devices 24. The transfer of the container blanks 12 to the movement devices 24 can, for example, be effected or assisted by a stroke movement of the respective movement device 24 relative to the base element 22. For example, the respective movement device 24 can be removed from the base element 22 at least temporarily in order to allow a holder of the movement device 24 to penetrate or enter the container blank 12 as a result of the stroke movement. The container blank 12 can thus be transferred from the feed conveyor 14. After the container blank 12 has been transferred, the movement device 24 can move closer to the base element 22 again with a stroke movement.

The (heating) furnace 18 is configured to heat the container blanks 12. The furnace 18 and the planar drive system 16 are so arranged relative to one another that the movement devices 24 can move the container blanks 12 through the furnace 18.

Preferably, the furnace 18 can have at least one heating channel 26 for heating the container blanks 12. The heating channel 26 can run in a straight line or can have a curved profile. The heating channel 26 can be equipped on at least one long side with heating segments, for example infrared heating elements, electric heating plates, microwave heaters or laser heaters. It is possible that the heating channel 26 also has reflectors on a side of the heating channel 26 opposite the heating segments.

The heating channel 26 can be open toward a longitudinal outer side (e.g. upper side, underside, left outer side, right outer side). The base element 22 is arranged on the open longitudinal outer side of the heating channel 26. The container blanks 12 can be moved through the heating channel 26 by the movement devices 24 through the open longitudinal outer side.

For example, the heating channel 26 can be open at an upper side. The base element 22 can be arranged above the heating channel 26. The movement devices 24 can be movable upside down on an underside of the base element 22. The movement devices 24 can move on the base element 22 outside the heating channel 26 and thereby move the container blanks 12 through the heating channel 26 through the open upper side.

Alternatively, the heating channel 26 can be open at an underside, for example. The base element 22 can be arranged beneath the heating channel 26. The movement devices 24 can be movable upright on an upper side of the base element 22. The movement devices 24 can move on the base element 22 outside the heating channel 26 and thereby move the container blanks 12 through the heating channel 26 through the open underside.

While the movement devices 24 are moving through the furnace 18 with the container blanks 12, the container blanks 12 can be rotated. Uniform heating of the container blanks 12 can thus preferably be achieved. The rotation can be effected by a rotation mechanism of the movement device 24 (see e.g. exemplary embodiment of FIG. 7). The rotation can also be effected by rotation of the movement devices 24, which are able to rotate relative to the base element 22 about their own vertical axis, for example while they are moving through the at least one heating channel 26. It is also possible that the rotation by means of the rotation mechanism and the rotation by means of the respective movement device 24 are superimposed on one another, for example in order in some portions to at least partially balance out/compensate for the movement effected by the rotation mechanism by the movement effected by means of the movement device 24. An optimum temperature distribution, for example, can thus be ensured, in particular in the case of so-called shaped bottles, etc.

The movement devices 24 move through the furnace 18 at equal distances. If the feed conveyor 14 transfers to the movement devices 24 a stream of container blanks 12 that is non-uniform at least in some portions, the movement devices 24 can be so moved relative to one another (e.g. acceleration, retardation, vary distances, etc.) that the distances between the container blanks 12 transported by the movement devices 24 are substantially equal again.

It is possible that one or more discharge conveyors (for example belt conveyor or transport starwheel) are arranged for transferring the container blanks 12 heated by the furnace 18 from the movement devices 24 and transporting them to the blow molding machine 20. Also in the case of the transfer to the discharge conveyor, the respective movement device 24 can perform a stroke movement relative to the base element 22 in order to effect or assist with the transfer, if desired. By means of the stroke movement, the holder can, for example, move out of or be removed from the heated container blank 12, for example while the heated blank is already held in another way by the discharge conveyor.

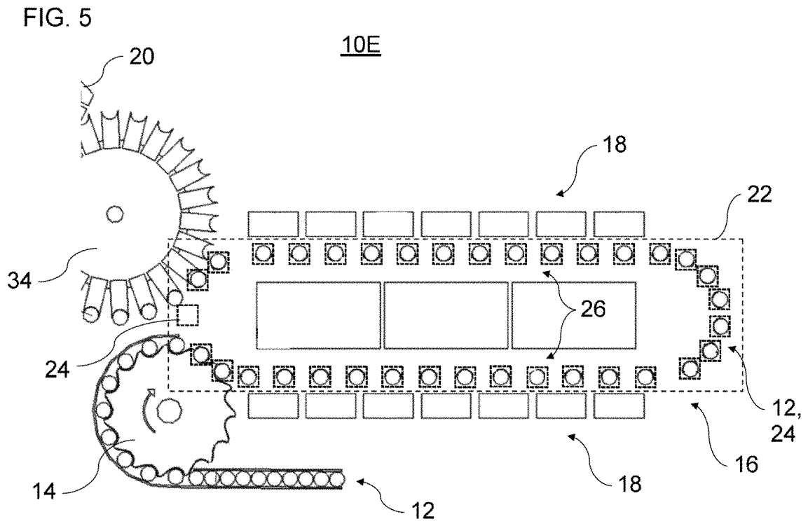

After the transfer of the heated container blank 12, the respective movement device 24 can be moved back to the feed conveyor 14 again. A further container blank 12 can be transferred to the feed conveyor 14. Preferably, the movement device 24 bypasses the furnace 18 on the way back to the feed conveyor 14. For example, the movement device 24 can be moved back next to the furnace 18 along the base element 22, for example along an edge region of the base element 22. It is also possible that an inlet and an outlet of the furnace 18 are arranged side by side (see e.g. exemplary embodiment of FIG. 5), which can shorten the return path significantly.

The blow molding machine 20 is arranged downstream of the furnace 18 and the planar drive system 16. In the blow molding machine 20, the container blanks 12 heated by the furnace 18 can be blow molded to form containers. The blow molding machine 20 can be in the form of, for example, a stretch blow molding machine, for example in carousel form. Alternatively, the blow molding machine 20 can, for example, be arranged stationarily and/or be of modular construction. Downstream of the blow molding machine 20 there can also be arranged, for example, a filler (e.g. filler carousel) for filling the containers and a capper for capping the containers, or also one or more coating stations for internally coating the container blanks 12 or containers.

Optionally, the device 10A can have a holder magazine 28. The holder magazine 28 can be arranged within reach of the movement devices 24, for example at an edge of the base element 22. The holder magazine 28 can store a plurality of different holders for container blanks 12 of different sizes. For example, the holders can be adapted to different neck/throat/mouth geometries of the container blanks 12. For a change of format, the movement devices 24 can be moved to the holder magazine 28. At the holder magazine 28, the current holder of the respective movement device 24 can automatically be replaced by a different holder. The holder can preferably be mounted on the respective movement device 24 or changed by means of a quick-change system, preferably a plug-, latch- and/or screw-type quick-change system. Preferably, the holder magazine 28 has a plurality of holders of each holder type, so that a plurality of movement devices 24 can change holder.

FIG. 2 shows a modified device 10B.

In the device 10B, the furnace 18 has a plurality of heating channels 26. The heating channels 26 are arranged, for example, parallel to one another. Depending on the throughput, the heating channels 26 can be switched on or switched off. For example, in the case of a maximum throughput, all the heating channels 26 can be activated. The container blanks 12 are transported by the movement devices 24 through all the heating channels 26. In the case of a minimum throughput, only one of the plurality of heating channels 26, for example, is activated. The container blanks 12 are transported by the movement devices 24 only through the activated heating channel 26. The remaining (inactive) heating channels 26 remain unused or can be used by the movement devices 24 on the return to the feed conveyor 14. It would also be possible to arrange a plurality of stationary blow molding stations or blow molding machines 20 after the individual heating channels.

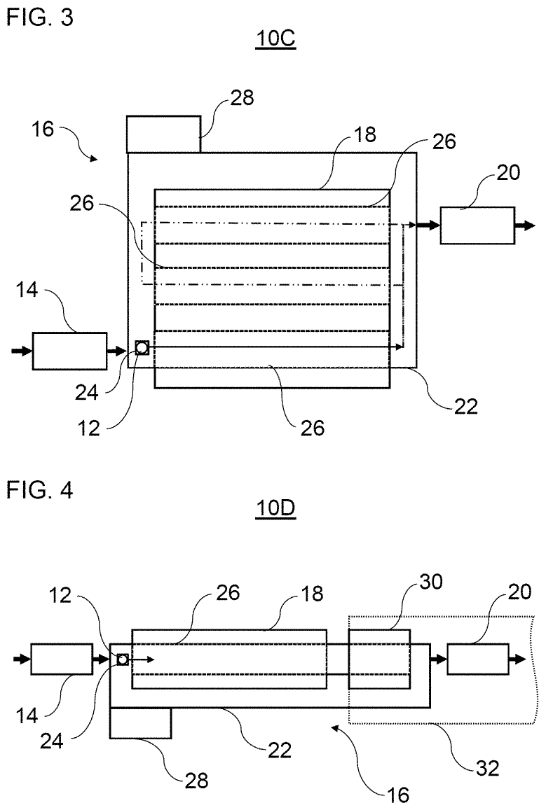

FIG. 3 shows a modified device 10C.

The device 10C differs from the device 10B in that the movement devices 24 can cover heating sections of different lengths through the furnace 18. For example, a short heating section can be chosen in the case of a small throughput. The short heating section passes through only one heating channel 26. In the case of a large throughput, on the other hand, a long heating section can be chosen, for example having two or more heating channels. A length of the heating section through the furnace 18 can thus be adapted by the movement devices 24, preferably in dependence on a current throughput. As the throughput increases, a heating section through the furnace 18 can be lengthened. Variable lengths of the heating section through the furnace 18 can alternatively or additionally also be provided by a plurality of exits along a heating channel 26. It is also possible that a movement speed of the movement devices 24 through the furnace 18 can be adapted in order to adapt a desired residence time in the furnace 18.

FIG. 4 shows a modified device 10D.

The device 10D has a sterilization apparatus 30. The sterilization apparatus 30 is arranged downstream of the furnace 18. The sterilization apparatus 30 is arranged upstream of the blow molding machine 20. The sterilization apparatus 30 is connected to the planar drive system 16. The movement devices 24 can be moved through the sterilization apparatus 30 with the transported container blanks 12. In the sterilization apparatus 30, the container blanks 12 can be sterilized. For example, the container blanks 12 can be treated with hydrogen peroxide or with electromagnetic radiation.

The device 10D can have a cleanroom 32. There can be arranged in the cleanroom 32, inter alia, the sterilization apparatus 30 and the blow molding machine 20 (as well as, for example, a filler and a capper). A (slight) excess pressure, for example, can prevail in the cleanroom in order to prevent an ingress of particles in the air. The cleanroom 32 can be connected to the planar drive system 16. The movement devices 24 can enter the cleanroom 32 and leave the cleanroom 32, for example through at least one airlock.

FIG. 5 shows a device 10E which discloses more structural details than the devices 10A to 10D.

The device 10E has a furnace 18 with a U-shaped heating channel 26. The base element 22 (shown in transparent form) is arranged above the furnace 18. The movement devices 24 (likewise shown in transparent form) move upside down on an underside of the base element 22. A feed conveyor 14 in the form of a sawtooth starwheel transfers the container blanks 12 to the movement devices 24. The movement devices 24 move the container blanks 12 through the heating channel 26. The movement devices 24 transfer the heated container blanks 12 to a discharge conveyor 34 in the form of a transport starwheel, which transfers the container blanks 12 to the blow molding machine 20 (shown only in part).

FIG. 6 shows an example of a movement device 24.

The movement device 24 is movable upside down on an underside (drive surface) of the base element 22. The movement device 24 is carried, preferably in a contactless manner, on the base element 22. The movement device 24 carries a holder 36 for the container blanks 12. The holder 36 is preferably in the form of an active or passive mandrel. The holder 36 can be inserted into an opening of a throat region or neck region of the container blank 12, for example by means of a stroke movement of the movement device 24 downward relative to the base element 22. The holder 36 can bear against multiple sides inside the throat region and thus hold the container blank 12. It is also possible that the holder 36 is of a different form and, for example, can engage around the throat region.

FIG. 7 shows a variant of the movement device 24 with a rotation mechanism 38.

The rotation mechanism 38 can be used to rotate the held container blank(s) 12 in the furnace 18. The rotation mechanism 38 allows the holder 36 and thus the container blank 12 to rotate without the movement device 24 rotating for that purpose. To that end, the rotation mechanism 38 interacts with a rotation apparatus 40. The rotation apparatus 40 can be part of the furnace 18. The rotation apparatus 48 can extend at least in some portions along the heating channel 26 of the furnace 18 (see e.g. FIG. 1).

The rotation mechanism 38 mounts the holder 36 in a rotatable manner on a base of the movement device 24. The rotation mechanism 38 can be, for example, an externally toothed rotary plate (for example toothed wheel) on which the holder 36 is mounted. The rotation apparatus 40 is stationary and configured such that it can interact with the rotation mechanism 38 in order to rotate the rotation mechanism 38 when the movement device 24 is moved sufficiently closely along the rotation apparatus 40. For example, the rotation apparatus 40 can be a toothed rack. The toothed rack can be straight, for example, as is shown in FIG. 7, or bent. The rotation mechanism 38 can mesh with the rotation apparatus 40 in order to rotate the rotation mechanism 38 and thus the holder 36.

It is possible that the rotation mechanism 38 and the rotation apparatus 40 are formed differently. For example, the rotation mechanism 38 could be in the form of a wheel or a roller, and the rotation apparatus 40 could be in the form of a rolling surface. In another example, an outer circumference of the rotation mechanism 38 in the form of a rotary plate is produced from a (ferro)magnetic material, and the rotation apparatus 40 has at least one permanent magnet, or vice versa. In principle, the rotation mechanism 38 can preferably be rotated by the rotation apparatus 40 by means of force-based engagement and/or by means of interlocking engagement when the movement device 24 moves along the rotation apparatus 40.

The invention is not limited to the preferred exemplary embodiments described above. Rather, a large number of variants and modifications are possible which likewise make use of the inventive concept and therefore fall within the scope of protection. In particular, the invention also claims protection for the subject matter and the features of the dependent claims independently of the claims to which they refer. In particular, the individual features of independent claim 1 are each disclosed independently of one another. In addition, the features of the dependent claims are also disclosed independently of all features of independent claim 1 and, for example, independently of the features relating to the presence and/or the configuration of the furnace and/or of the planar drive system of independent claim 1.

### LIST OF REFERENCE NUMERALS

10A-10E device for treating container blanks
12 container blank
14 feed conveyor
16 planar drive system
18 furnace
20 blow molding machine
22 base element
24 movement device
26 heating channel
28 holder magazine
30 sterilization apparatus
32 cleanroom
34 discharge conveyor
36 holder
38 rotation mechanism
40 rotation apparatus

1-15. (canceled)

16. A device for treating container blanks for a container treatment installation, having:
a furnace for heating the container blanks; and
a planar drive system having a base element and a plurality of movement devices for transporting the container blanks,
wherein the plurality of movement devices are movable independently of one another relative to the base element, and
the container blanks can be moved through the furnace via the plurality of movement devices.

17. The device of claim 16, wherein the plurality of movement devices are movable independently of one another relative to the base element via magnetic interaction between the base element and the plurality of movement devices.

18. The device of claim 16, wherein one of:
the base element is arranged above the furnace, and the plurality of movement devices can be moved upside down on an underside of the base element; and
the base element is arranged beneath the furnace, and the plurality of movement devices can be moved upright on an upper side of the base element.

19. The device of claim 16, wherein:
the furnace has at least one heating channel which is open toward a longitudinal outer side; and
the base element is arranged on the longitudinal outer side.

20. The device of claim 19, wherein the base element is arranged on the longitudinal outer side for moving the container blanks through the heating channel via the plurality of movement devices.

21. The device of claim 16, wherein the plurality of movement devices each have at least one holder.

22. The device of claim 21, wherein the at least one holder is a mandrel configured to hold at least one container blank at a neck region of the container blank.

23. The device of claim 21, wherein at least one of:
the at least one holder is designed to be changeable; and
the device has a holder magazine which holds a plurality of different holders, and the plurality of movement devices can be moved to the holder magazine in order to change the at least one holder.

24. The device of claim 23, wherein the at least one holder is rotatable via a rotation mechanism of the respective movement device in order to rotate the at least one held container blank while it is being moved through the furnace.

25. The device of claim 24, wherein the furnace has a rotation apparatus, which is configured to rotate the rotation mechanism while the respective movement device is being moved along the rotation apparatus.

26. The device of claim 25, wherein the rotation apparatus is configured to rotate the rotation mechanism via at least one of an interlocking engagement and a force-based engagement.

27. The device of claim 25, wherein one of:
the rotation mechanism has a first toothed portion and the rotation apparatus has a second toothed portion which mesh with one another in order to rotate the rotation mechanism;
the rotation mechanism has a roller and the rotation apparatus has a rolling surface for the roller; and
the rotation mechanism interacts with the rotation apparatus via magnetic force in order to rotate the rotation mechanism.

28. The device of claim 27, wherein the first toothed portion is a toothed wheel and the second toothed portion is a toothed rack.

29. The device of claim 16, wherein the planar drive system is configured to rotate the plurality of movement devices relative to the base element about their own vertical axis while the container blanks are being moved through the furnace.

30. The device of claim 16, wherein the planar drive system is configured to carry out a stroke movement of the movement device relative to the base element one of when a container blank is transferred to a movement device and when a container blank is transferred from a movement device.

31. The device of claim 16, wherein the planar drive system is configured to move a stream of container blanks transferred at non-uniform distances to the plurality of movement devices through the furnace at a uniform distance.

32. The device of claim 16, wherein at least one of:
the planar drive system is configured to adapt a length of a heating section of the movement of the container blanks through the furnace via the plurality of movement devices; and
the planar drive system is configured to adapt a residence time of the container blanks in the furnace by adapting a speed of the plurality of movement devices.

33. The device of claim 16, wherein the furnace has a plurality of individually activatable heating channels through which the container blanks are movable in a flexible manner via the plurality of movement devices in dependence on a respective desired length of a heating section of the movement of the container blanks through the furnace.

34. The device of claim 16, further comprising:
a sterilization apparatus for sterilizing the container blanks,
wherein the container blanks can be moved through the sterilization apparatus via the plurality of movement devices.

35. The device of claim 16, further comprising:
a blow molding machine for blow molding containers from the container blanks,
wherein at least one of: the blow molding machine is arranged downstream of the furnace, and the blow molding machine is configured for the transfer of container blanks from the plurality of movement devices.
